(19) United States
(12) Patent Application Publication
(10) Pub. No.: US 2013/0059020 A1
(43) Pub. Date: Mar. 7, 2013

(54) COSMETIC ACTIVE INGREDIENT PREPARATION FOR INCREASING THE LONG-TERM EPIDERMAL VITALITY OF THE SKIN

(71) Applicants: MARCUS ASAM, Unterfoehring (DE); MIRJAM ASAM, Unterfoehring (DE)

(72) Inventors: MARCUS ASAM, Unterfoehring (DE); MIRJAM ASAM, Unterfoehring (DE)

(21) Appl. No.: 13/599,530

(22) Filed: Aug. 30, 2012

(30) Foreign Application Priority Data
Sep. 2, 2011 (DE) 10 2011 112 092.4

(51) Int. Cl. A61K 8/97; A61Q 19/08; A61K 38/02

(52) U.S. Cl. 424/766; 514/1.1

(57) ABSTRACT

A cosmetic active ingredient preparation serves to increase the long-term epidermal vitality and reduce the wrinkle depth of a skin. The active ingredient preparation has at least 18% of a grape seed extract and at least 10% of a grape shoot and/or grape peel extract. Moreover, the active ingredient preparation comprises at least 0.5% of a polysaccharide and at least 0.0005% of an iron peptide.

The present application claims priority to German patent application number: DE 10 2011 112 092.4, filed Sep. 2, 2011.

### BACKGROUND OF THE INVENTION

1. Field of the invention

This invention relates to a cosmetic active ingredient preparation for increasing the long-term epidermal vitality of the skin.

2. Description of prior art

It is known that external environmental influences such as, for example, UVB radiation, ionizing radiation such as X-ray radiation, gamma radiation and beta radiation, and also various chemical substances damage the skin. The epidermis of the skin can repair this damage to a certain extent. However, if the damage is too great, inflammatory reactions result, especially in the epidermis, which considerably impair the appearance of the skin and, in the long-term, lead to increased wrinkling.

It is known from WO 02/080875 A2 that retinol in cosmetic active ingredient preparations has a long-lasting antiwrinkle effect and also a radical- scavenging effect. However, retinol is relatively unstable, meaning that it can develop its advantageous effect only for a very short time. For this reason, this document proposes using retinyl palmitate instead of retinol, which accordingly stabilizes the retinyl radical. In the meantime, retinyl palmitate has established itself as an agent of first choice for cosmetic products with an anti-ageing or wrinkle-depth-reducing effect. Besides the particular suitability for ageing skin, vitamin A is known for aiding the regeneration of UV-damaged skin and improving the natural skin functions.

### BRIEF SUMMARY OF THE INVENTION

The object of the invention is to provide a cosmetic active ingredient preparation for increasing the long-term epidermal vitality of the skin coupled with simultaneous reduction in wrinkles.

This object is achieved with the following features.

A cosmetic active ingredient preparation has at least 18% of a grape seed extract and at least 10% of a grape shoot and/or grape peel extract. These extracts are often used in cosmetic products and have in particular a skin-smoothing and also anti-ageing effect. Moreover, this cosmetic active ingredient preparation comprises at least 0.5% of a polysaccharide. This has a high water storage effect and can accordingly introduce correspondingly large amounts of water into the skin. Consequently, this extract likewise has a skin-smoothing effect, with inflammatory reactions being additionally reduced. Finally, the cosmetic active ingredient preparation also comprises at least 0.005% of an iron peptide. Iron is actually known as an inflammation-promoting substance. Nevertheless, it has been established that in the presence in particular of the grape seed extract in the cosmetic active ingredient preparation, the iron peptide has a significantly greater regeneration effect than retinyl palmitate. Although retinyl palmitate is more rapid in its regenerative effect than iron peptide, after just 48 hours, in the case of iron peptide in the presence of the grape seed extract, a significantly greater regenerative effect was demonstrated. This is all the more surprising as the iron peptide in itself has no regenerative effect at all but, on the contrary, is even slightly inflammation-promoting. The precise chemical mechanism of action which leads to this surprising result is currently unexplained.

It is advantageous when the grape seed extract comprises oligomeric polyphenol. This substance acts as radical scavenger and therefore protects the skin against environmental influences.

If the grape shoot and/or grape peel extract comprises resveratrol, then an advantageous anti-ageing effect arises.

To achieve a particularly good water storage effect, the polysaccharide comprises mannose, xylose and/or glucuronic acid, preferably combined in the polymer.

It is favourable when the iron peptide has at least one of the amino acids histidine, alanine, threonine and isoleucine. These amino acids are required in particular for building up the connective tissue structure in the epidermis and therefore promote skin regeneration.

A cosmetic preparation comprises the above-described cosmetic active ingredient preparation and at least one fatty acid. This facilitates application to the skin and gives it a pleasant consistency.

For this fatty acid a linoleic acid and/or octanoic acid in triglyceride form has proven useful, which can be readily absorbed by the skin.

To adjust the pH, the preparation comprises at least one organic acid and/or inorganic hydroxide solution with a carbon chain which has at least three carbon atoms. Consequently, this organic acid is sufficiently weak in order itself not to serve as a trigger of inflammatory reactions.

It is advantageous when the organic acid is a hydroxypropanetricarboxylic acid. This acid is of natural origin and sufficiently skin-compatible. If the pH is too low, sodium hydroxide solution which is likewise skin-compatible is used to adjust the pH.

Further advantages and features of the present invention are presented in the following detailed description with reference to the accompanying figure which contains an exemplary embodiment of the present invention. It should be understood, however, that the drawing serves merely for the purpose of illustrating the invention and does not restrict the scope of protection of the invention.

### DETAILED DESCRIPTION OF THE INVENTION

Test formulation 1

A test formulation 1 according to the patent consists of:
88.88% of water,
   6.00% of methylpropanediol as solvent,
   3.00% of caprylic acid in triglyceride form,
   0.70% of inulin as carrier material,
   0.482485% of glycerol,
   0.10% of a sodium glutamate as emulsifier,
   0.02% of citric acid,
   0.0025% of phenoxyethanol as preservative and
   0.8150515% of an active ingredient preparation.

This active ingredient preparation comprises:
61% of grape seed extract,
37% of grape shoot extract,
   1.998% of polysaccharide and
   0.002% of iron hexapeptide.

To investigate test formulation 1, a full skin model was created which is a human, bioartificial skin fragment. This consists of a sponge-like structured collagen matrix in which human dermal fibroblasts have been sown. An epidermis equivalent consisting of human keratinocytes lies on this artificial, but living, dermis. This full skin model is cultivated under optimized conditions although it contains no skin pigments since no melanocytes of any kind are present. Since skin pigments are known to offer very effective UV protection, this full skin model reacts in a particularly sensitive way to UVB radiation.

These full skin models were mildly irritated using UVB radiation. This means that the radiation dose was chosen to be so weak that no lasting apoptotic change in the tissue is visible. The total radiation dose introduced was 40 mJ/cm² in a wavelength range between 290 nm and 320 nm.

This irradiation, especially with UVB light, produces predamage of the skin models, which brings about certain inflammatory reactions in the skin model. Using the skin model compared with real subjects has the advantage in particular that exactly defined experimental conditions are present, especially since the skin condition before the start of application of the test formulation is defined as flawless. This gives rise to reliable statements concerning the effectiveness of different test formulations.

Both 24 hours and 48 hours after applying the test formulation to the predamaged skin model, the secretion of the human pro-inflammatory cytokine interleukin-6 in the skin model was analysed. This value indicates an inflammatory reaction in the skin model and should in the ideal case be as low as possible. 24 hours after applying test formulation 1, this interleukin-6 value was 13 000 pg/ml. 48 hours after applying test formulation 1, the interleukin-6 value reached 7000 pg/ml.

These two values were compared with two untreated skin models. In comparison sample A, the skin model was not predamaged in any way, such that the native interleukin-6 value is ascertained. This was about 5000 pg/ml. In the case of a test sample B, on the other hand, the skin model was predamaged in the same way as for test formulation 1 by means of UV rays, without a regenerative formulation being applied. After hours, the interleukin-6 value was 15 000 pg/ml, which dropped to about 10 000 pg/ml 48 hours after predamage. Accordingly, test formulation 1 shows an interleukin-6 value which, especially 48 hours after applying test formulation 1, is closer to the non-predamaged sample than to the untreated but predamaged sample. Accordingly, it was shown that more than half of the inflammatory reactions caused by the UV irradiation have subsided as a result of using test formulation 1. Particularly if one considered that iron actually has an inflammation-promoting effect, this result is surprising.

Test formulation 2

This active ingredient preparation comprises:
89.38% of water,
   6.00% of methylpropanediol as solvent,
   3.00% of caprylic acid in triglyceride form,
   0.70% of inulin as carrier material,
   0.10% of a sodium glutamate as emulsifier,
   0.02% of citric acid, and
   0.80% of an active ingredient preparation.

This active ingredient preparation comprises:
62.5% of grape seed extract, and
37.5% of grape shoot extract.

This test formulation 2 serves as reference for test formulation 1 since here the addition of the iron peptide in particular has been dispensed with. Test formulation 2 was investigated in the same way as test formulation 1 in a corresponding full skin model and, in so doing, again the interleukin-6 value after 24 hours and after 48 hours was ascertained. The interleukin-6 value was about 13 000 pg/ml after 24 hours and about 10 000 pg/ml after 48 hours. This value is significantly above that of test formulation 1, meaning that a significant, inflammation-inhibiting contribution by the iron peptide is demonstrated.

Test formulation 3

This active ingredient preparation comprises:
88.79% of water,
   6.00% of methylpropanediol as solvent,
   3.00% of caprylic acid in triglyceride form,
   0.70% of inulin as carrier material,
   0.482485% of glycerol,
   0.10% of a sodium glutamate as emulsifier,
   0.01% of citric acid,
   0.0025% of phenoxyethanol as preservative,
   0.000002% of tocopherol for stabilizing the retinyl palmitate and
   0.915013% of an active ingredient preparation.

This active ingredient preparation comprises:
54.64% of grape seed extract,
32.79% of grape shoot extract,
10.93% of retinyl palmitate,
   1.638% of polysaccharide and
   0.002% of iron hexapeptide.

Test formulation 3 differs from test formulation 1 in particular by virtue of the significant addition of ca. 11% of retinyl palmitate. Retinyl palmitate is used in dermatology for regenerating photodamaged skin. It is used in many cosmetic products. Test formulation 3 was investigated in the same way as test formulation 1. This gave interleukin-6 values of 8000 pg/ml after 24 hours and about 11 000 pg/ml after 48 hours.

The very good interleukin-6 value after 24 hours is entirely understandable and shows the known, favourable regenerating effect of retinyl palmitate. However, the effect of this regenerating active ingredient is only of extremely short duration and is virtually exhausted after just 48 hours. In particular, a significantly smaller regenerating effect than in the case of test formulation 1 arises after 48 hours. This result is surprising and could not be explained by the current understanding of the average person skilled in the art. In particular, it reveals that retinyl palmitate is unable to regenerate the predamaged epidermis in the long-term.

Test formulation 4
89.27% of water,
   6.00% of methylpropanediol as solvent,
   3.00% of caprylic acid in triglyceride form,
   0.70% of inulin as carrier material,
   0.10% of a sodium glutamate as emulsifier,
   0.03% of citric acid,
   0.000002% of tocopherol and
   0.899998% of an active ingredient preparation.

This active ingredient preparation comprises:
55.55% of grape seed extract,
33.33% of grape shoot extract and
11.12% of retinyl palmitate.

Test formulation 4 differs from test formulation 3 essentially by virtue of the omission of the iron peptide. Consequently, test formulation 4 shows the effect of retinyl palmitate without the presence of the iron peptide.

Test formulation 4 was again investigated in the same way as test formulation 1. 24 hours after applying test formulation 4, the interleukin-6 value was 12 000 pg/ml. 48 hours after application of test formulation 4, it was 11 000 pg/ml. This shows that the retinyl palmitate, especially in the case of the long-term effect, is disadvantageous 48 hours after applying the test formulation and leads to an interleukin-6 value which is close to the untreated but predamaged skin model. It is therefore shown that in the case of the long-term effect, the iron peptide together with the grape seed extract and the grape shoot and/or grape peel extract displays the best effect. By contrast, the usually used retinyl palmitate produces no kind of effect 48 hours after application and/or even impairs the effect of the iron peptide.

Wrinkle depth reduction

Test formulation 1 was additionally investigated with regard to its effect on the depth of wrinkles. These investigations were carried out on 30 subjects each with a healthy skin type. For all of the subjects, a wrinkle-depth measurement was carried out in accordance with Rz-DIN before, 24 hours after and 4 weeks after applying the test formulation 1. In this way, it is possible to ascertain both the short-term and the long-term reduction in wrinkle depth. The test formulation was applied twice daily to the test area.

The results are summarized in the table below.

| | Rz values | Change after | Change after |
|---|---|---|---|
| Subject | beforehand | 24 h | 4 weeks |
| No. | [μm] | [%] | [%] |
| 1. | 162.6 | −22.45 | −37.95 |
| 2. | 150.8 | −24.93 | −46.22 |
| 3. | 140.0 | −21.71 | −51.86 |
| 4. | 157.9 | −19.82 | −43.83 |
| 5. | 147.3 | −19.01 | −49.08 |
| 6. | 155.8 | −24.97 | −44.80 |
| 7. | 171.1 | −22.21 | −37.11 |
| 8. | 138.8 | −27.52 | −49.14 |
| 9. | 152.9 | −23.61 | −42.84 |
| 10. | 141.2 | −16.43 | −42.28 |
| 11. | 142.3 | −20.45 | −48.84 |
| 12. | 138.6 | −24.17 | −50.36 |
| 13. | 146.2 | −17.37 | −49.59 |
| 14. | 137.0 | −26.13 | −49.78 |
| 15. | 157.0 | −17.58 | −42.23 |
| 16. | 154.2 | −15.63 | −42.67 |
| 17. | 142.9 | −28.13 | −46.12 |
| 18. | 150.8 | −20.09 | −58.69 |
| 19. | 144.4 | −22.92 | −45.01 |
| 20. | 159.5 | −21.07 | −43.26 |
| 21. | 151.5 | −26.34 | −46.07 |
| 22. | 162.1 | −17.64 | −38.93 |
| 23. | 142.3 | −28.60 | −50.25 |
| 24. | 162.2 | −20.65 | −44.39 |
| 25. | 183.5 | −27.14 | −40.16 |
| 26. | 167.7 | −22.00 | −41.74 |
| 27. | 153.1 | −17.37 | −51.01 |
| 28. | 142.0 | −20.14 | −50.21 |
| 29. | 156.2 | −22.73 | −42.89 |
| 30. | 144.5 | −21.87 | −52.73 |

The 30 test persons tolerated the test formulation without objection in the four-week application test according to dermatological-clinical criteria. In no case did undesired or even pathological skin changes arise in the region of the test area. Consequently, undesired skin reactions or in particular skin-irritating effects can be excluded. The skin roughness and thus the wrinkle depth were measured using a 3D measurement of the skin surface, a special algorithm being used to find the area again. This ensures that all measurement values were ascertained from the exact same skin area. An average improvement in skin roughness after 24 hours with only one application of 22% was found. After carrying out the entire four-week application, the improvement was 45.8%. These surprisingly good values demonstrate the applicability of the test formulation as antiwrinkle cream.

Since some exemplary embodiments of the present invention are not shown or described, it must be understood that a multiplicity of changes and modifications of this exemplary embodiment described are possible, without departing from the essential idea and scope of protection of the invention defined by the claims.

1. Cosmetic active ingredient preparation for increasing the long-term epidermal vitality and reducing the wrinkle depth of a skin, wherein the active ingredient preparation comprises at least 18% of a grape seed extract, at least 10% of a grape extract derived from at least one of a grape shoot and grape peel at least 0.5% of a polysaccharide, at least 0.0005% of an iron peptide, and at least 10% of a grape extract derived from at least one of a grape shoot and grape peel.

2. Cosmetic active ingredient preparation according claim 1, wherein said grape seed extract comprises oligomeric polyphenol.

3. Cosmetic active ingredient preparation according to claim 1, wherein said grape extract derived from at least one of grape shoot and grape peel comprises resveratrol.

4. Cosmetic active ingredient preparation according to claim 1, wherein said polysaccharide comprises at least one of the monomers mannose, xylose and glucuronic acid.

5. Cosmetic active ingredient preparation according to claim 1, wherein said iron peptide comprising at least one of amino acids alanine, histidine, isoleucine and threonine.

6. Cosmetic preparation with at least 0.005% of a cosmetic active ingredient preparation for increasing the long-term epidermal vitality and reducing the wrinkle depth of a skin, wherein the active ingredient preparation comprises at least 18% of a grape seed extract, at least 10% of a grape extract derived from at least one of a grape shoot and grape peel at least 0.5% of a polysaccharide, at least 0.0005% of an iron peptide, and at least 10% of a grape extract derived from at least one of a grape shoot and grape peel and said cosmetic preparation comprises at least one fatty acid.

7. Cosmetic preparation according to claim 6, wherein said fatty acid comprises at least one of linoleic acid and octanoic acid in triglyceride form.

8. Cosmetic preparation with at least 0.005% of a cosmetic active ingredient preparation for increasing the long-term epidermal vitality and reducing the wrinkle depth of a skin, wherein the active ingredient preparation comprises at least 18% of a grape seed extract, at least 10% of a grape extract derived from at least one of a grape shoot and grape peel at least 0.5% of a polysaccharide, at least 0.0005% of an iron peptide, and at least 10% of a grape extract derived from at least one of a grape shoot and grape peel and said cosmetic preparation comprising at least one of an organic acid with a carbon chain having at least three carbon atoms and an inorganic hydroxide solution.

9. Cosmetic preparation according to claim 8, wherein said organic acid is a hydroxypropanetricarboxylic acid.

10. Cosmetic preparation according to claim 8, wherein said inorganic hydroxide solution is sodium hydroxide solution.
